(19) United States
(12) Patent Application Publication
(10) Pub. No.: US 2026/0157697 A1
(43) Pub. Date: Jun. 11, 2026

(54) SYSTEM, METHOD, AND APPARATUS FOR PROVIDING REAL TIME BIOLOGICAL SIGNAL MONITORING IN A WEARABLE DEVICE

(71) Applicant: The Johns Hopkins University, Baltimore, MD (US)

(72) Inventors: Francesco V. Tenore, Fulton, MD (US); Abby R. Joseph, Lexington Park, MD (US); Robert D. Stevens, Owings Mills, MD (US); Ralph Etienne-Cummings, Washington, DC (US); John Rattray, Norcross, GA (US)

(73) Assignee: The Johns Hopkins University, Baltimore, MD (US)

(21) Appl. No.: 19/342,500

(22) Filed: Sep. 27, 2025

Related U.S. Application Data
(60) Provisional application No. 63/728,195, filed on Dec. 5, 2024.

(51) Int. Cl. A61B 5/00; A61B 5/0205; A61B 5/021; A61B 5/024; A61B 5/0531; A61B 5/11; A61B 5/318; A61B 5/389; G16H 40/67; G16H 50/30

(52) U.S. Cl. CPC A61B 5/6804; A61B 5/0205; A61B 5/0531; A61B 5/1126; A61B 5/318; A61B 5/389; G16H 40/67; G16H 50/30; A61B 5/021; A61B 5/02416; A61B 2562/0219; A61B 2562/06

(57) ABSTRACT

A method for monitoring a biological signal in a wearable context includes receiving first sensor data from a first sensor including a first electrode and a second electrode, the first sensor being disposed proximate to a chest of a wearer, receiving second sensor data from a second sensor including a third electrode and a fourth electrode, the second sensor being disposed proximate a distal end of a limb of the wearer, synchronizing the first sensor data and the second sensor data to extract signal data from the first and second sensor data, performing feature extraction on the first and second sensor data to estimate the biological signal based on comparing results of the feature extraction to a model, and storing a continuous record of the biological signal.

### CROSS-REFERENCE TO RELATED APPLICATION

This application claims priority to and the benefit of U.S. Provisional Application No. 63/728,195 filed on Dec. 5, 2024, the entire contents of which are hereby incorporated herein by reference.

### TECHNICAL FIELD

Example embodiments generally relate to techniques for biological signal monitoring and, in particular, relate to devices that can provide continuous monitoring of such signals in a wearable context.

### BACKGROUND

Wearable devices (e.g., watches, rings, bracelets, patches, etc.) that monitor various biological signals are not new. Such devices exist today in numerous contexts, and attempt to measure numerous different types of biological signals. However, certain biological signals may be more difficult to measure than others, particularly if the goal is to perform monitoring using non-invasive wearable devices. Blood pressure is one example of such a biological signal.

Blood pressure is typically measured using the very familiar cuff, which is attached to a patient's arm in a very overt and specific measurement effort. The patient is typically asked to sit and relax during the measurement. This provides a consistent milepost by which to monitor blood pressure for the patient over various discretely measured data points gathered at intervals in time. It does not provide monitoring for all of the other time, and through all of the other various activities the patient will normally engage in on a daily basis. Moreover, this type of measurement actually takes many tens of seconds to obtain, and is not an instantaneous measurement in any case.

This example of monitoring a biological signal on an intermittent and episodic basis is merely one case where a continuous record of data for the biological signal may be helpful. However, even in the age of wearables, no solution for doing so has yet been put forth given the limitations on things like battery life, sensor capability, and practical matters like where and how to measure certain biological signals. Example embodiments provide a comprehensive solution to overcome the substantial limitations noted above.

### BRIEF SUMMARY

Some non-limiting, example embodiments include a system that including a wearable and multi-modal sensor array that can acquire real-time data for certain biological signals (like blood pressure) in a non-invasive package, and on a continuous basis.

In one example embodiment, an apparatus for monitoring a biological signal in a wearable context may be provided. The apparatus may include processing circuitry configured to execute instructions that, when executed, cause the apparatus to perform various operations. The operations may include receiving first sensor data from a first sensor including a first electrode and a second electrode, the first sensor being disposed proximate to a chest of a wearer, receiving second sensor data from a second sensor including a third electrode and a fourth electrode, the second sensor being disposed proximate a distal end of a limb of the wearer, synchronizing the first sensor data and the second sensor data to extract signal data from the first and second sensor data, performing feature extraction on the first and second sensor data to estimate the biological signal based on comparing results of the feature extraction to a model, and storing a continuous record of the biological signal.

In another example embodiment, a method for monitoring a biological signal in a wearable context may be provided. The method may include receiving first sensor data from a first sensor including a first electrode and a second electrode, the first sensor being disposed proximate to a chest of a wearer, receiving second sensor data from a second sensor including a third electrode and a fourth electrode, the second sensor being disposed proximate a distal end of a limb of the wearer, synchronizing the first sensor data and the second sensor data to extract signal data from the first and second sensor data, performing feature extraction on the first and second sensor data to estimate the biological signal based on comparing results of the feature extraction to a model, and storing a continuous record of the biological signal.

In still another example embodiment, a system for monitoring a biological signal in a wearable context may be provided. The system may include a first sensor including a first electrode and a second electrode disposed proximate to a chest of a wearer to obtain first sensor data, a second sensor including a third electrode and a fourth electrode disposed proximate a distal end of a limb of the wearer to obtain second sensor data, and a monitoring device wirelessly operably coupled to the first sensor and the second sensor to receive the first sensor data and the second sensor data. The monitoring device may be time-synchronized with the first and second sensors and include processing circuitry configured to extract signal data from the first and second sensor data. The monitoring device performs feature extraction on the first and second sensor data to estimate the biological signal based on comparing results of the feature extraction to a model. A continuous record of the biological signal is stored by the monitoring device.

### BRIEF DESCRIPTION OF THE SEVERAL VIEWS OF THE DRAWINGS

Having thus described the invention in general terms, reference will now be made to the accompanying drawings, which are not necessarily drawn to scale, and wherein:

FIG. 1 illustrates a functional block diagram of a system that may be useful in connection with continuously monitoring a biological signal in a wearable context according to an example embodiment;

FIG. 2 illustrates a functional block diagram of a monitoring device according to an example embodiment;

FIG. 3 illustrates a functional block diagram of two sensors in communication with the monitoring device in accordance with an example embodiment;

FIG. 4 illustrates a demonstration sensor placement on the body in accordance with an example embodiment;

FIG. 5 illustrates a garment that may be used to position sensors on the body when the garment is worn in accordance with an example embodiment;

FIG. 6 illustrates a block diagram of various steps that may be employed in connection with generating a model for use in accordance with an example embodiment;

FIG. 7 illustrates a plot of data points associated with measuring a biological signal to determine pulse transit time (PTT) in accordance with an example embodiment;

FIG. 8 illustrates a plot of data points associated with measuring a biological signal to determine pulse arrival time (PAT) in accordance with an example embodiment;

FIG. 9 illustrates a plot of data points associated with measuring a biological signal to determine pre-ejection period (PEP) in accordance with an example embodiment;

FIG. 10 illustrates a display screen of a user interface at a monitoring device in accordance with an example embodiment; and

FIG. 11 illustrates a block diagram of a method for monitoring a biological signal in a wearable context according to an example embodiment.

### DETAILED DESCRIPTION

Some non-limiting, example embodiments now will be described more fully hereinafter with reference to the accompanying drawings, in which some, but not all example embodiments are shown. Indeed, the examples described and pictured herein should not be construed as being limiting as to the scope, applicability or configuration of the present disclosure. Rather, these example embodiments are provided so that this disclosure will satisfy applicable legal requirements. As used herein, operable coupling should be understood to relate to direct or indirect connection that, in either case, enables functional interconnection of components that are operably coupled to each other. Like reference numerals refer to like elements throughout.

As noted above, remote monitoring of biological signals using wearables is already conducted in certain relatively limited scenarios. However, the scope of health monitoring is often limited to only measurement of fairly basic parameters. Furthermore, the ability to perform continuous monitoring is also often limited by battery life. To provide a non-invasive continuous monitoring device, and particularly such a device that may be capable of monitoring more complex biosignals, i.e., ones that require access to more than one sensor, such as blood pressure, example embodiments provide specific hardware and software components as enabling technologies. As an example, example embodiments may provide a wearable array of multi-modal sensors that provide continuous, real-time monitoring of biological signals. The sensors may be placed at multiple sites across the body to monitor local biological signal activity as well as the propagation of signals between measurement sites. Example embodiments may employ many different modalities including, for example, photoplethysmography, electrodermal activity, electromyography, electrocardiography, 3-axis accelerometry, and acoustic. Devices in the system may be synchronized to each other to allow measurements of signals that depend on temporal differences between the original source signals. Moreover, the system of an example embodiment may be configurable into different combinations of active sensors, on a single device or across multiple devices in a network. Multiple combinations can also be achieved depending upon the precise placement of sensors, and where they are located in a measurement network.

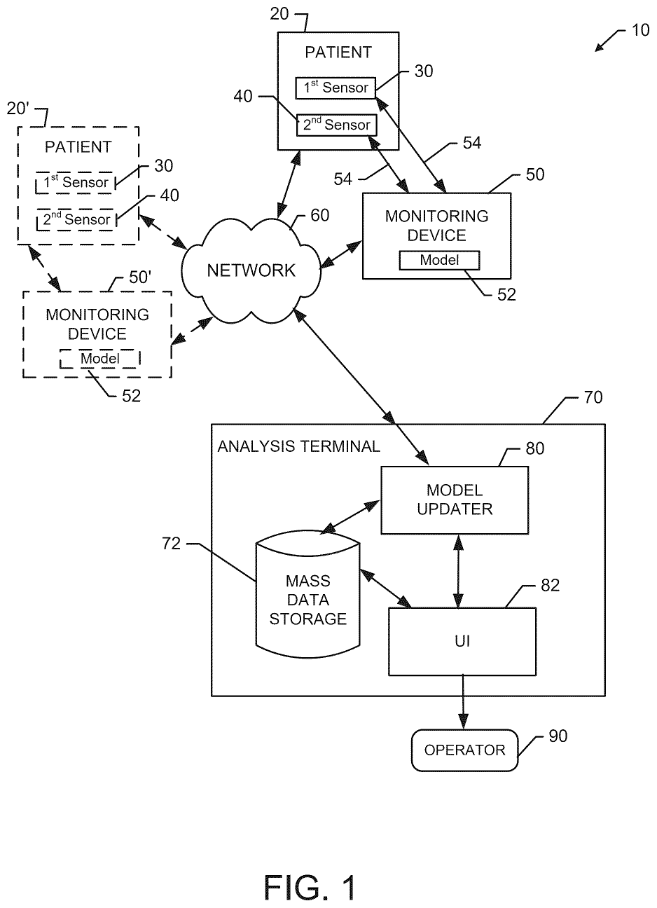

FIG. 1 illustrates a functional block diagram of a system 10 that may be useful in connection with continuously monitoring a biological signal in a wearable context according to an example embodiment. In this regard, as shown in FIG. 1, the system 10 may include components that are wearable by a patient 20 (or multiple patients, as indicated by the existence of a second patient 20′ in FIG. 1). The system 10 may include a first sensor 30 and a second sensor 40, each of which are wearable components that are worn by the patient 20. The first and second sensors 30 and 40 may be operably coupled to a monitoring device 50 that includes a model 52 that is trained to enable the data that is measured at the first and second sensors 30 and 40 to be converted into estimates of the corresponding biosignal (e.g., blood pressure) in real-time. Of note, a second monitoring device 50′ is also shown in connection with the second patient 20′ to illustrate the potential for multiplicity of patients, monitoring devices, and sensors that may form parts of the system 10. However, it should be appreciated that the system 10 may be fully functional with as little as one patient (and one set of the first and second sensors 30 and 40), or a multitude of patients (and respective instances of first and second sensors 30 and 40).

In an example embodiment, the first and second sensors 30 and 40 may each be configured to communicate with the monitoring device 50 to share raw data sensed or measured at the first and second sensors 30 and 40, respectively, with the monitoring device 50. The communication may be direct (e.g., via a wireless connection that may be proprietary or open) or may be indirect (e.g., via a network 60). In some examples, to keep power consumption relatively low, a BLUETOOTH® Low Energy (BLE) link 54 may be utilized for communication between the first and second sensors 30 and 40 and the monitoring device 50. The network 60, when used, may include a wireless communication access point such as a WIFI® router, or the like that may operably couple one or more instances of the monitoring device 50 (and or sensors associated therewith) to an analysis terminal 70, which may be remotely located.

In some cases, the analysis terminal 70 may be a remote server, which may include enhanced capability processing and storage resources that may consume massive amounts of data from a potential multitude of patients and monitoring devices to permit not only storage of such data on massive scales (e.g., via mass data storage 72), but to also permit a model updater 80 to perform updates to the model 52 over time, as described in greater detail below. The analysis terminal 70 may also include a user interface (UI), which may allow an operator 90 to interact with the analysis terminal 70 with respect to model training and updating, and for dissemination of models to respective instances of the monitoring device 50 after the model 52 has been identified or otherwise selected to be changed, replaced or updated. In some cases, the analysis terminal 70 may also provide calibration services for the monitoring device 50. For example, when docked for recharging, in some cases, the analysis terminal 70 may provide calibration testing and updating for the monitoring device 50. However, the monitoring device 50 may also include locally executable instructions for calibration in some cases. Calibration may occur at relatively large intervals including at least 24 hours and, in some cases, longer periods than that.

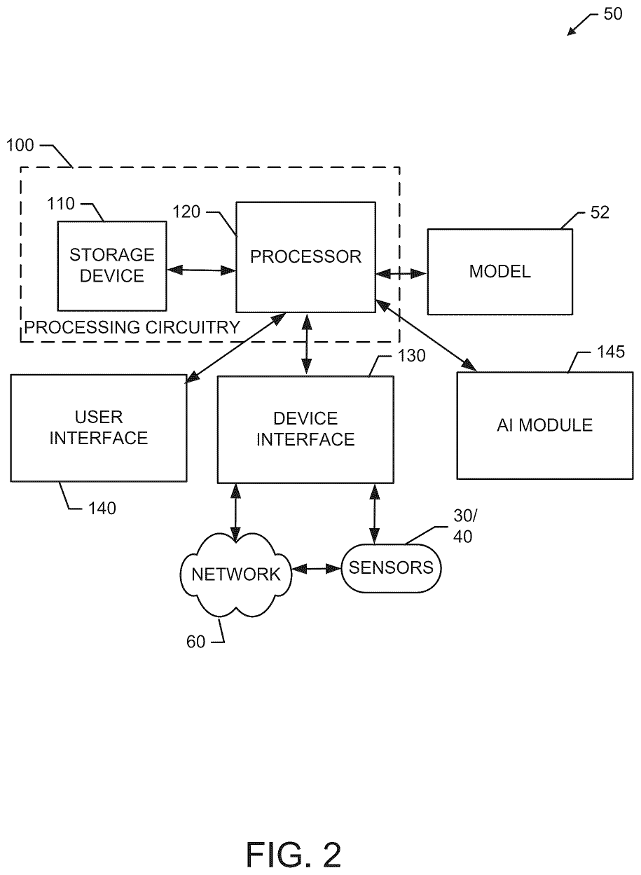

FIG. 2 provides a more detailed block diagram view of the monitoring device 50 of an example embodiment. As shown in FIG. 2, the monitoring device 50 may include processing circuitry 100 that is configured to perform data processing, application execution and other processing and management services according to an example embodiment of the present invention. In one embodiment, the processing circuitry 100 may include a storage device 110 and a processor 120 that may be in communication with or otherwise control a user interface 140 and a device interface 130. As such, the processing circuitry 100 may be embodied as a circuit chip (e.g., an integrated circuit chip) configured (e.g., with hardware, software or a combination of hardware and software) to perform operations described herein. However, in some embodiments, the processing circuitry 100 may be embodied as a portion of a server, computer, laptop, workstation, cellular phone, tablet or even one of various other mobile computing devices. In situations where the processing circuitry 100 is embodied as a server or at a remotely located computing device, the user interface 140 may be disposed at another device that may be in communication with the processing circuitry 100 via the device interface 130 and/or a network (e.g., network 60).

The user interface 140 may be in communication with the processing circuitry 100 to receive an indication of a user input at the user interface 140 and/or to provide an audible, visual, mechanical or other output to the user (e.g., alerts or output data). As such, the user interface 140 may include, for example, a keyboard, a mouse, a joystick, a display, a touch screen, a microphone, a speaker, or other input/output mechanisms. In some cases, the user interface 140 may also include a series of web pages or interface consoles generated to guide the user through various options, commands, flow paths and/or the like for control of or interaction with the monitoring device 50. The user interface 140 may also include interface consoles or message generation capabilities to send instructions, alerts, notices, etc., and/or to provide an output to a user (e.g., the patient 20, or another party including, for example, the operator 90).

The device interface 130 may include one or more interface mechanisms for enabling communication with other devices and/or networks. In some cases, the device interface 130 may be any means such as a device or circuitry embodied in either hardware, software, or a combination of hardware and software that is configured to receive and/or transmit data from/to a network and/or any other device or module in communication with the processing circuitry 100. In this regard, the device interface 130 may include, for example, hardware and/or software for enabling communications with a wireless communication network and/or a communication modem or other hardware/software for supporting communication via cable, digital subscriber line (DSL), universal serial bus (USB), Ethernet or other methods. In situations where the device interface 130 communicates with a network, the network may be any of various examples of wireless or wired communication networks such as, for example, data networks like a Local Area Network (LAN), a Metropolitan Area Network (MAN), and/or a Wide Area Network (WAN), such as the Internet. As noted above, the device interface 130 may also include antennas and/or the like to facilitate wireless communication via WIFI®, BLUETOOTH® or other relatively short range communication protocols. However, proprietary communication protocols, and even long range communication protocols (e.g., 5G) may alternatively be employed in some cases.

In an example embodiment, the storage device 110 may include one or more non-transitory storage or memory devices such as, for example, volatile and/or non-volatile memory that may be either fixed or removable. The storage device 110 may be configured to store information, data, applications, instructions or the like for enabling the apparatus to carry out various functions in accordance with example embodiments of the present invention. For example, the storage device 110 could be configured to buffer input data for processing by the processor 120. Additionally or alternatively, the storage device 110 could be configured to store instructions for execution by the processor 120. As yet another alternative, the storage device 110 may include one of a plurality of databases that may store a variety of files, contents or data sets such as the raw biosignal data measured by the first and second sensors 30 and 40. Among the contents of the storage device 110, applications may be stored for execution by the processor 120 in order to carry out the functionality associated with each respective application.

The processor 120 may be embodied in a number of different ways. For example, the processor 120 may be embodied as various processing means such as a microprocessor or other processing element, a coprocessor, a controller or various other computing or processing devices including integrated circuits such as, for example, an ASIC (application specific integrated circuit), an FPGA (field programmable gate array), a hardware accelerator, or the like. In an example embodiment, the processor 120 may be configured to execute instructions stored in the storage device 110 or otherwise accessible to the processor 120. As such, whether configured by hardware or software methods, or by a combination thereof, the processor 120 may represent an entity (e.g., physically embodied in circuitry) capable of performing operations according to embodiments of the present invention while configured accordingly. Thus, for example, when the processor 120 is embodied as an ASIC, FPGA or the like, the processor 120 may be specifically configured hardware for conducting the operations described herein. Alternatively, as another example, when the processor 120 is embodied as an executor of software instructions, the instructions may specifically configure the processor 120 to perform the operations described herein.

In an example embodiment, the processor 120 (or the processing circuitry 100) may be embodied as, include or otherwise control the application of data received from the first and second sensors 30 and 40 to the model 52 to generate an estimate of a biological parameter that is to be monitored by the monitoring device 50 such as, for example, blood pressure. Thus, the monitoring device 50 may be any means such as a device or circuitry operating in accordance with software or otherwise embodied in hardware or a combination of hardware and software (e.g., processor 120 operating under software control, the processor 120 embodied as an ASIC or FPGA specifically configured to perform the operations described herein, or a combination thereof) thereby configuring the device or circuitry to perform the corresponding functions of the monitoring device 50 (or components thereof) as described herein.

The conversion of raw data measured by the first and second sensors 30 and 40 to an estimate of a biological signal (e.g., biosignal) or biological parameter (such as blood pressure) that is performed by the monitoring device 50 is reliant on, and only as accurate as, the model 52. Thus, it can be appreciated that the initial formulation, training and structuring of the model 52 may be an important aspect of example embodiments. The ability to train and update the model 52, especially as more data is obtained, and more information is learned, may therefore also be an important aspect of example embodiments. The network 60 may therefore enable (e.g., via the device interface 130) updating of the model 52 when updates are generated by the model updater 80.

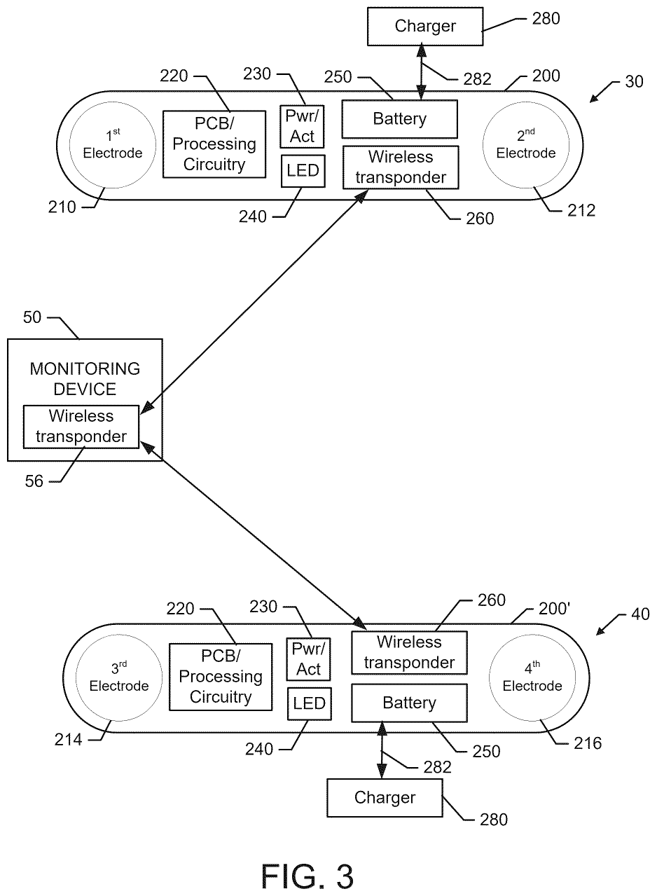

The first and second sensors 30 and 40 may, in some cases, be structurally identical, but may be placed or worn at different parts of the body of the patient 20. However, in some cases, particularly due to the different locations that the first and second sensors 30 and 40 may be worn, the structure may be modified to fit the circumstances of the location worn. Regardless of any potential structural differences, the functional characteristics may be similar and are represented generally by the block diagram of FIG. 3. In some cases, the first and second sensors 30 and 40 may perform measurements on data that may benefit from further analysis using artificial intelligence (AI) such as, for example a generative pre-trained transformer (GPT) large language model (LLM), etc. Thus, for example, the monitoring device 50 may include an AI module 145 to provide such analysis. However, as an alternative, the AI module 145 may be instantiated at the analysis terminal 70, or any other suitable place in the network 60.

Turning now to FIG. 3, the first and second sensors 30 and 40 may each include a body portion 200 and 200′, respectively. The body portion 200 and 200′ may, as noted above, either be identical or different, but may generally provide a physical structure and form factor of a housing inside which electronic and other components of the first and second sensors 30 and 40 may be housed. The first sensor 30 may include a first electrode 210 and a second electrode 212, and the second sensor 40 may include a third electrode 214 and a fourth electrode 216. The first, second, third and fourth electrodes 210, 212, 214 and 216 may be structurally and/or functionally identical, and may be disposed on opposing longitudinal ends of the body portion 200 and 200′, respectively, in order to provide physical distance therebetween to permit measurement of parameters between the two spaced apart locations of each respective sensor. However, it should be appreciated that parameters may be measured and compared also between electrodes of different sensors as well. In an example embodiment, the body portion 200 may have a length as small as 40 mm, a width as small as 13 mm, and a height as small as 8 mm. Overall weight of the first and second sensors 30 and 40 may vary, but some embodiments may weigh as little as 8 grams.

The first, second, third and fourth electrodes 210, 212, 214 and 216 may be operably coupled to a printed circuit board (PCB) and/or processing circuitry 220 of each respective sensor. The first, second, third and fourth electrodes 210, 212, 214 and 216 may, in some cases, be embodied as MAX30101 electrodes by Maxim. Thus, for example, the electrodes may have the capability of measuring reflectance in infrared ranges (e.g., wavelength of 880) with a sampling frequency of about 250 Hz and resolution of 18 bits. The PCB and/or processing circuitry 220 may include one or more PCBs with corresponding circuitry (e.g., processor and memory) and circuit chips (e.g., ASIC/FPGA) that functionally enable the respective sensors to operate in the manner described herein. Various chips and components of the PCB and/or processing circuitry 220 may have sampling frequencies, resolutions, gains and other properties selected to enhance performance of their respective tasks. For example, sampling frequencies from 25 to 250 Hz may be provided in some cases. The first and second sensors 30 and 40 may have a limited user interface, which may include a function button such as, for example, a power/action button 230 and a display, which may be limited to the point of including one or more instances of a light emitting diode (LED) 240. Each of the first and second sensors 30 and 40 may have its own respective instance of a battery 250, which may be replaceable or rechargeable. In some cases, the battery 250 may be a LiPo rechargeable battery.

To facilitate the wireless communication described above, each of the first and second sensors 30 and 40 may also include a wireless transponder 260, which may be configured to communicate with a wireless transponder 56 of the monitoring device 50. As noted above, the wireless transponders 260 and 56 may employ BLUETOOTH®, WIFI®, or other protocols. However, in an example embodiment, BLE may be employed in order to maximize life of the battery 250. However, other examples may employ other low energy communications protocols such as, for example, ZIGBEE®, Ultra Wide Band (UWB) impulse radios, etc. A runtime of at least 24 hours may be achievable, and still provide continuous monitoring for the entire period for which coverage is provided.

Although the battery 250 of some embodiments may be replaceable, some example embodiments may configure the battery 250 to be rechargeable in order to minimize the difficulty of maintenance. When the battery 250 is rechargeable, a charger 280 may be configured to be operably coupled to the battery 250 via a charging interface 282. In some examples, the first and second sensors 30 and 40 may generally be worn all day, and they may be docked at night on the charger 280. When docked, the charging interface 282 may be aligned to permit recharging of the battery 250 while the patient 20 is sleeping. However, other charging strategies may alternatively be employed including, for example, wireless charging that may enable the first and second sensors 30 and 40 to be powered essentially continuously, since recharging may be conducted without the patient 20 removing the first and second sensors 30 and 40.

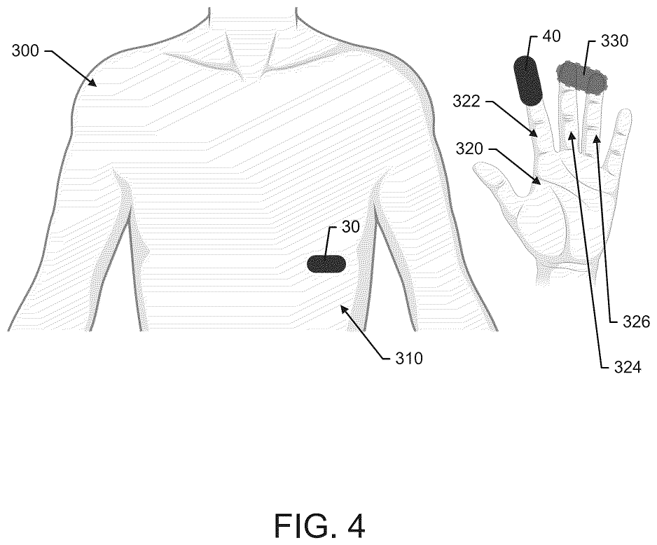

The first and second sensors 30 and 40 may be placed on different parts of a body 300 of the patient 20, as shown in FIG. 4. The specific locations for placement on the body 300 may be strategically selected based on the parameters that are ultimately desired to be measured and/or estimated. In the example case of measuring blood pressure, the first sensor 30 may be selected for placement proximate to a heart of the patient (e.g., at a chest portion 310 of the body 300). Meanwhile, the second sensor 40 may be selected for placement proximate to a distal end of one of the limbs of the patient 20. In the depicted example, a hand 320 of the patient 20 is shown, and the distal end of the hand 320 is actually near the tip of the index finger 322. Any other finger could alternatively be used, however. Moreover, in some cases, it should be appreciated that more than just the first and second sensors 30 and 40 may be employed. FIG. 4 illustrates this possibility by showing the middle finger 324 and ring finger 326 of the hand further having a third sensor 330 provided thereon. One of the electrodes of the third sensor 330 may be in contact with the middle finger 324, and the other electrode of the third sensor 330 may be in contact with the ring finger 326. Meanwhile, both electrodes of the second sensor 40 are in contact with the index finger 322. It should be appreciated that toes could be substituted for fingers, if desired, and even wrist, ankle or other relatively distally located portions of the limbs could also be used. In some cases, setup of the system may include the patient 20 (or operator 90) defining the locations of the sensors specifically. However, in other cases, the monitoring device 50 may be configured to determine the locations of the sensors without being specifically told (e.g., based on measurements by the sensors).

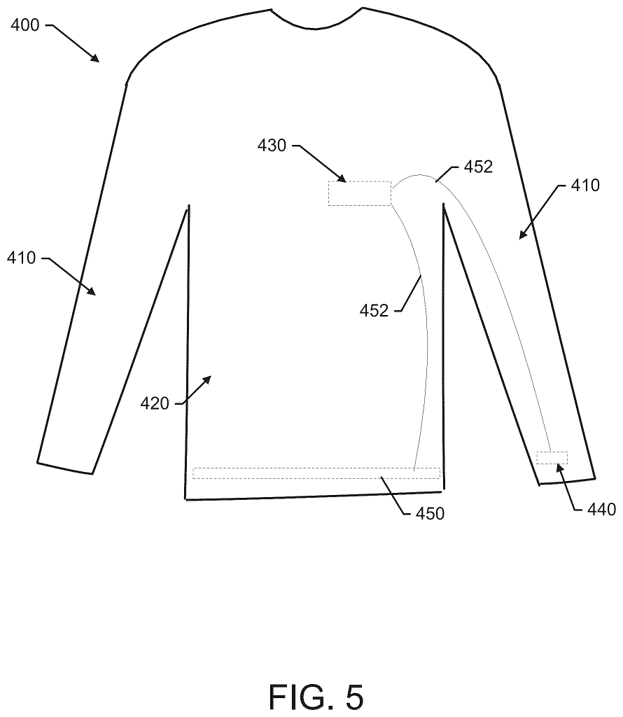

In an example embodiment, an adhesive may be used to attach (or stick) the first, second and third sensors 30, 40 and 330 to the body 300 at their respective mounting locations. The adhesive may, for example, be on the same side of the body portion 200 and 200′ of the respective sensors as the electrodes, to ensure contact between the electrodes and the skin. However, other methods of fixation may be used in some cases. For example, straps (e.g., with hook and loop fasteners, snaps, buckles and/or the like) may be used to wrap around the chest, fingers, toes, etc., to affix the corresponding sensors in location. Another example method of ensuring proper location of the sensors may be to provide a garment 400 that is specifically constructed to include locating pockets or mounts for the sensors. FIG. 5 shows an example of one such instance of the garment 400 that may serve in this capacity.

Turning to FIG. 5, the garment 400 may include sleeves 410 into which arms of the patient 20 may be inserted, and a front portion 420 that may lie proximate to the chest portion 310 of the body 300 when worn. A distal end of the sleeves 410 may be referred to as a cuff portion, and the cuff portion may naturally lie proximate a wrist of the patient 20 when the garment 400 is worn on the body 300. The garment 400 may be provided with a chest mount 430 at the front portion 420 configured to hold the first sensor 30 proximate the chest portion 310. The chest mount 430 may be a pocket, hook and loop faster patch, or other mounting structure on an interior side of the front portion 420 that interfaces with a side of the first sensor 30 that is opposite the first and second electrodes 210 and 212 to place the first and second electrodes 210 and 212 next to the skin when the garment 400 is worn. The garment 400 may also be provided with a cuff mount 440, which may be located at a distal end of one of the sleeves 410. The cuff mount 440 may be a pocket, hook and loop faster patch, or other mounting structure on an interior side of the sleeve 410 that interfaces with a side of the second sensor 40 that is opposite the third and fourth electrodes 214 and 216 to place the third and fourth electrodes 214 and 216 next to the skin when the garment 400 is worn.

In addition to more or less automatically positioning the first and second sensors 30 and 40 at desirable locations, the garment 400 may also be augmented to incorporate improved battery performance and longer life of the sensors. In this regard, for example, a battery mount 450 may be provided in some cases to enable an external battery to be housed at the garment 400. The external battery supported at the battery mount 450 may provide a battery that can provide power directly to the first and second sensors 30 and 40 (instead of internal batteries such as battery 250), or act as a charging battery to charge the battery 250. Internal wiring 452 may be provided to enable operable coupling of the external battery in the battery mount 450 to the first and second sensors 30 and 40 at the chest mount 430 and the cuff mount 440, respectively. In an example embodiment, the battery mount 450 may be distributed over a large surface area in order to allow a distributed battery structure to be employed to permit a large number of cells, and therefore electrical capacity to be provided without concentrating the weight and size all at a single location. The distributed battery may therefore provide even greater capability for real-time and continuous monitoring as described herein.

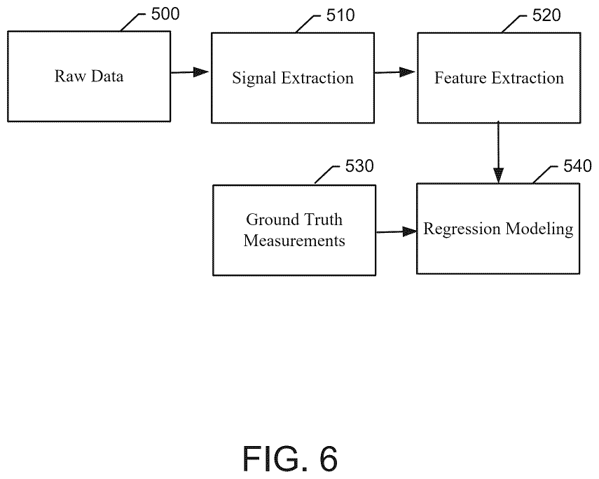

As noted above, the model 52 may be trained to perform estimations of a particular biological parameter or signal based on measurement of other (typically more accessible) parameter or signal measurements. Thus, for example, various precursor parameters may be measured and the model 52 may provide a transformation from the precursor parameters into the biological parameter or signal that is the ultimate goal or target parameter for monitoring via estimation of the target parameter based on measurement of its precursor parameters. In the case of blood pressure, the precursor parameters or signals may include, for example, pulse transit time (PTT), pulse arrival time (PAT), and pre-ejection period (PEP). To achieve an accurate estimate of the target parameter, a process similar to that shown in reference to FIG. 6 may be employed.

As shown in FIG. 6, an initial measurement of raw data 500 may be performed with respect to the precursor parameters that will be measured to provide the basis for an estimation of another biological signal that is related to the precursor parameters. Thereafter, signal extraction 510 may performed on the raw data 500. In some cases, the raw data 500 may include electrocardiogram (ECG) data and photoplethysmography (PPG) data extracted based on the sensor data obtained via the first and second sensors 30 and 40. In an example embodiment, the raw data 500 may further include accelerometry, electrodermal activity and electromyography data among other potential parameters. During signal extraction 510, the signals from the raw data that correlate to ECG, PPG, accelerometry, electrodermal activity and/or electromyography may be extracted using vector extraction or other applicable techniques. Thereafter, feature extraction 520 may be performed prior to feeding the extracted feature data into a regression model for regression modeling 530. The regression modeling 530 may also receive ground truth measurements of the parameter that is to be estimated so that, for example, the model 52 may be constructed to map the extracted features associated with the precursor parameters to ground truth measurements. In the case of blood pressure, ground truth blood pressure measurements (e.g., made with a conventional blood pressure measurement cuff) may be compared to corresponding ECG, PPG and accelerometry data to define respective mappings therebetween within the model 52.

In an example embodiment, the model 52 may be built using one or more different regression models. Various example embodiments have employed as many as twenty eight different regression models to date. Of those twenty eight different regression models, good performance has been noted particularly with respect to rational quadratic Gaussian Process Regression (GPR), a quadratic Support Vector Machine (SVM), a Fine Tree or a linear SVM. Although the model 52 may be built according to any selected regression model, fusion of results from different models may also be employed in some cases.

It should also be noted that example embodiments are aimed at providing continuous monitoring, which may occur across many different activity states of the patient 20 instead of only the normal seated and relaxed measurement of blood pressure that is typically done with a blood pressure cuff on nearly every medical related visit that people have. Accordingly, given that blood pressure may be related to the precursor parameters differently for different states of activity, the model 52 may also be structured to include portions thereof that relate to different states of activity. Thus, for example, the model 52 may be built for taking into account data that is segmented across various situations or conditions that may correlate precursors to blood pressure estimates differently. As it relates to activity, sitting, deep breathing, Valsalva, posture change, mental computation, breath holding, stair climbing, etc. may each be measured and modeled separately. Accelerometry data or various other techniques may then be used by the monitoring device 50 to estimate which activity the patient 20 is engaged in during period over which measurements of the precursor parameters are being obtained. Corresponding portions of the model 52 that relate to the same activity may then be used to obtain blood pressure estimates.

Of note, activity is just one such differentiating situation that may impact modeling accuracy. Others differentiators may include age, weight, height, gender, race, or any other category where a particular correlation may exist between patients fitting into the category and how the precursors typically map to estimated blood pressure for the category on a statistical basis. These other differentiators may be obtained via profile information or medical record information about the patient 20, or via directly inquiring. When the patient 20 has provided all information about himself/herself that can be relevant to selecting the best model, the analysis terminal 70 may select the best model for the patient 20 and send it to the monitoring device 50 via the network 60. The selected best model (e.g., model 52) may then be used for the patient 20 with respect to all estimates being performed for the patient 20. However, it should be appreciated that the second patient 20′ may have an entirely different model provided thereto, as may each and every other patent being monitored. As such, each model may be tailored to the patient 20, but may also include specific portions of the model 52 that correspond to respective different activities that the patient 20 may be engaged in at any given time.

In an example embodiment, the analysis terminal 70 and/or the monitoring device 50 may incorporate artificial intelligence (AI) tools (e.g., AI module 145) to facilitate further/deeper analysis of the data that is recorded (e.g., locally at the monitoring device 50 or in the mass data storage 72). Thus, for example, by wearing the sensor for long periods of the time (e.g., 3 months), the continuously measured data (e.g., continuous BP monitoring) may allow data trends (e.g., a “Blood Pressure Trend” (BPT) to be computed to provide a percentage of time that the wearer is in hypertensive excursion, hypotensive excursion, or experiencing various peaks, valleys, etc. relative to parameters measured or estimated. These trends and analyses may generate a Stroke Risk Score or a Blood Pressure related Stroke Risk Score for the wearer. In effect, the data collected and the continuous blood pressure readings generated and stored may provide a wealth of biomarker information that may be analyzed for future correlations to diseases, conditions and/or hazardous situations that may be based not only on biomarkers that are not just based on biological samples, but based on data. Both short term and long term trends, and data over short and long term timeframes, may therefore be analyzed for deeper patterns and relations to health care related outcomes to improve (e.g., via learning) identification and treatment options over time.

Additionally, in some cases, the AI module 145 may be configured to make complex inferences or determinations based only on individual sensor data, but the temporal and spatial relationships between the sensors (i.e., nodes), such as phase differences, signal propagation delays, or correlated patterns. The temporal and spatial relationships, and data associated therewith, may be useful to provide valuable insights into a plethora of physiological processes, system dynamics, and overall health status. Data from medical records or otherwise entered into the system 10 by the operator 90, the patient 20, or any other party, may also be contributory to the analyses. The use of AI for these determinations can therefore be exceptionally useful not only in the short term, but in terms of adapting the capabilities of the system 10 as time moves forward, and more data from more diverse sources is obtained.

As noted above, the model 52 may be initially generated based on the process described in reference to FIG. 6, and may be updated at various intervals thereafter. The intervals may be based on time or data volume. Thus, for example, updates may occur weekly, monthly, annually or at some other time-based interval, or updates may occur whenever predefined volume thresholds are reached that suggest enough significant data may be possessed to consider making an update. Also, if any significant medical changes occur that result in changes to the profile of the patient 20 or are evident in a linked medical record of the patient 20, model changes or updates may be driven by the change, or when the operator 90 notices the change and manually triggers a corresponding update.

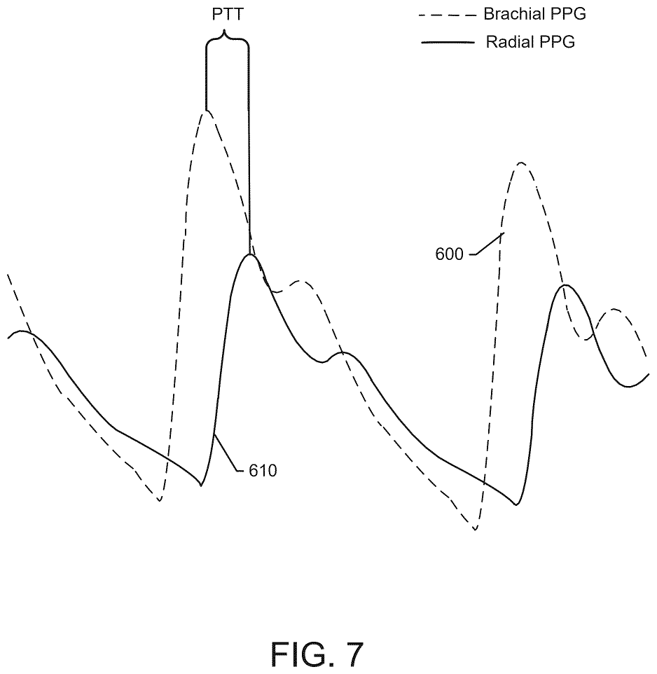

PTT, which is the time it takes for a pulse to travel between two arterial sites, may be one biological signal that is related to blood pressure, and can be measured relatively easily in order to be used in estimating blood pressure. In this regard, PTT is inversely correlated to blood pressure. PPG is a non-invasive optical technique for detecting changes in blood volume. Thus, measuring radial and brachial PPG may provide an opportunity to measure PTT, and thereby also get useful information for estimating blood pressure. FIG. 7 illustrates a plot showing a measurement of brachial PPG 600 and radial PPG 610. When the sensors (e.g., the first and second sensors 30 and 40) are properly synchronized, the peak volume measured can be compared to determine a time difference in a pulse wave, which represents the PPT, as shown in FIG. 7.

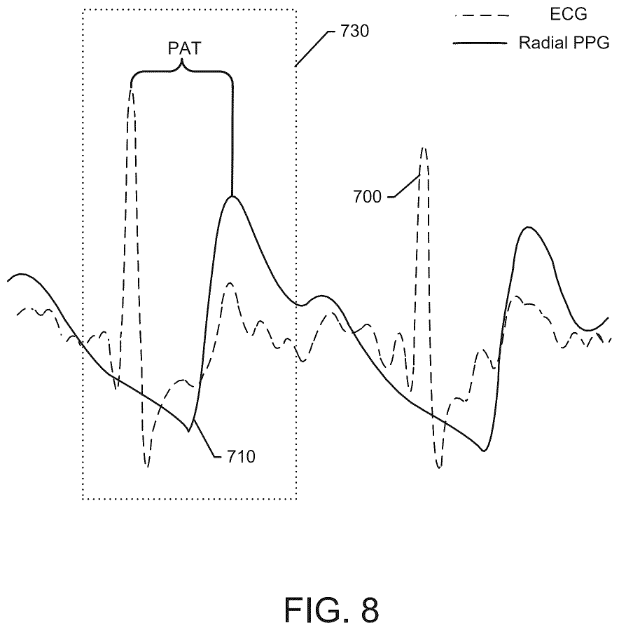

PAT is another measurable biological signal that is related to blood pressure, and therefore may be measured to estimate blood pressure. PAT is the time between electrical activation of the heart and the corresponding pulse wave at an arterial site. Thus, PAT is inversely correlated to blood pressure. ECG is a relatively easy to measure parameter that monitors the electrical activity of the heart. Relating ECG to PPG may therefore provide a means by which to measure PAT. FIG. 8 shows an example relating ECG 700 to radial PPG 710 measured and synchronized. The PAT is shown as the difference between respective peak measurements in each of the ECG 700 and radial PPG 710.

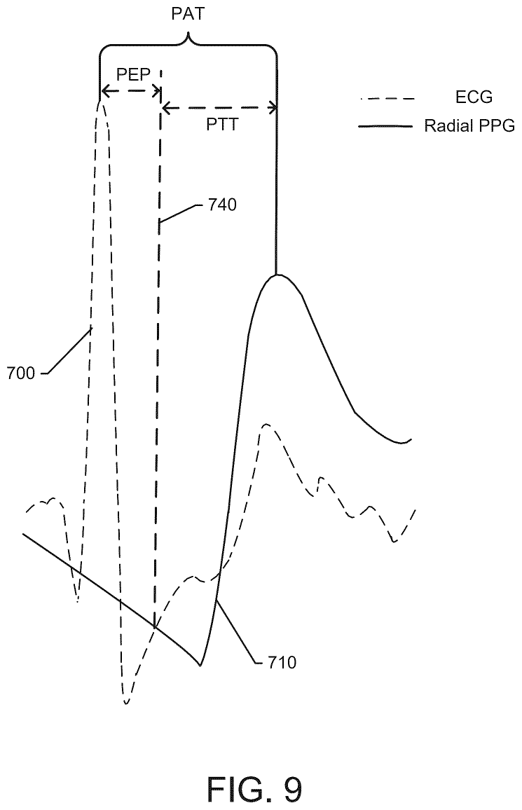

PEP, which measures the time between electrical activation of the heart (signal) and ventricular ejection (action) is generally not correlated with blood pressure, but changes with stress and physical activity. Thus, PEP may also be useful in detecting different stress related or physical activity related situations, which may be helpful in selecting models or portions of models that may be employed for blood pressure estimation at various times. To demonstrate how PEP may be determined, an enlarged section 730 of the plot in FIG. 8 is reproduced in greater detail in FIG. 9. Thus, again both the ECG 700 and radial PPG 710 are shown along with the point of ventricular ejection 740. The difference between the start of the PAT measurement and the ventricular ejection 740 is PEP. The remaining portion of the PAT, which is measured from the ventricular ejection 740 to the end of the PAT measurement is representative of PTT.

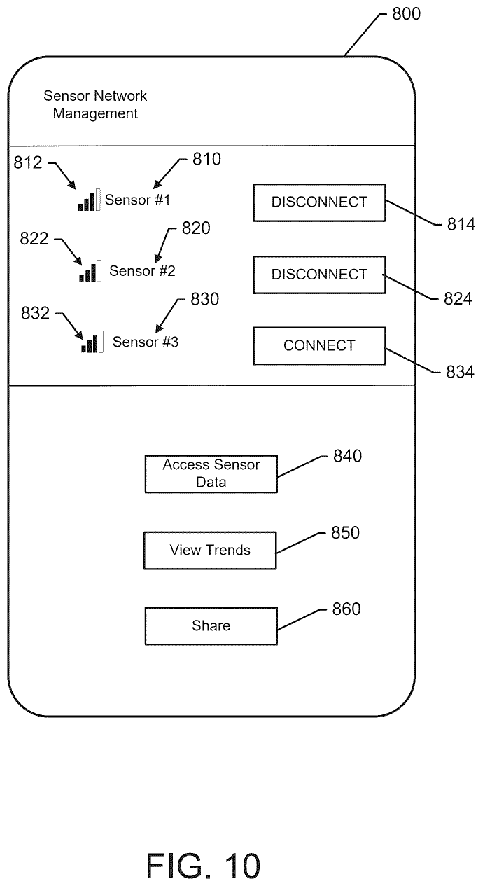

Synchronization is an important aspect to making sure accurate results can be achieved. To achieve such synchronization, the network 60 and/or the monitoring device 50 may be configured to achieve less than 10⁻⁶ sec synchronization between the sensors and the monitoring device 50. In an example embodiment, the first and second sensors 30 and 40 may be configured to synchronize to each other independently of any external devices. Thus, the monitoring device 50 receives data asynchronously. By providing independent synchronization, example embodiments may facilitate the integration of edge computing in future iterations where a compact model may run on the sensors (having small memory storage onboard) and the monitoring device 50 may only be used to visualize the data, provide longer term offline storage, and facilitate data transmission to network storage, etc. In addition to synchronization across all networked devices, which may be managed at the monitoring device 50 in some cases, the monitoring device 50 may also provide a capability for changing the configurations of sensors and/or electrodes of the sensors in order to permit measurements across electrodes and sensors in precisely determinable combinations. FIG. 10 illustrates an example interface including a display screen or control console 800 that may be generated at the monitoring device 50 to provide configuration changes. In this regard, as shown in FIG. 10, a first sensor 810, second sensor 820 and third sensor 830 may each be configurable into different combinations of active sensors. To facilitate decision making regarding desirable combinations, it may be helpful to appreciate the quality of the network connection for each respective one of the sensors. To facilitate this, the control console 800 may further display connectivity information for each sensor. As shown in FIG. 10, a first signal strength indicator 812 may be provided in association with the first sensor 810. Similarly, a second signal strength indicator 822 may be provided in association with the second sensor 820 and a third signal strength indicator 832 may be provided in association with the third sensor 830.

Connection selectors may then also be provided to enable the user (e.g., patient 20, operator 90 or other user) to disconnect any connected sensors or connect any disconnected sensors. In the example shown, the first and second sensors 810 and 820 are currently connected. A first connection selector 814 or a second connection selector 824 may therefore be selected to disconnect the first and second sensors 810 and 820, respectively. Meanwhile, the third sensor 830 is currently disconnected. A third connection selector 834 may be selected to connect the third sensor 830 to the first and second sensors 810 and 820. As noted above, in some cases it may also be possible to select individual electrodes from different sensors in order to define specific electrode combinations.

The control console 800 may also provide other functionalities, or at least links or selectors that enable access to other functionalities. As an example, an access sensor data button 840 may be provided to enable the viewing of live or recorded sensor data from an specific sensor, of a specific data type, or over a specific timeframe. A view trends button 850 may be provided to illustrate trend lines for raw data, precursor parameters, and/or the estimated biological signal or parameter that is the target parameter. Other options, such as a share button 860 may be provided to enable the monitoring device 50 to upload information to a doctor or medical team, to another device, or any other authorized recipient. Still other functions may be added in connection with some example embodiments.

Example embodiments may therefore provide a system of multi-modal sensors that can be used to measure biological signals of various kinds that can be used to estimate another biological signal for continuous monitoring in a non-invasive way. A specific example for calculating an estimate of blood pressure is shown, but the same principles may be applied to estimation of other biosignals as well.

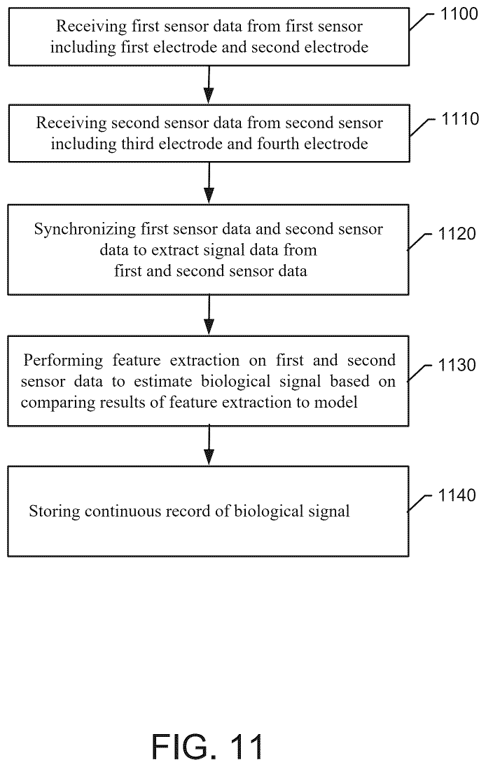

From a technical perspective, the monitoring device 50 described above in reference to FIG. 1 may be used to support some or all of the operations described above. As such, the platform or system described in FIG. 1 may be used to facilitate the implementation of several computer program and/or network communication based interactions. As an example, FIG. 11 is a flowchart of a method and program product according to an example embodiment of the invention. It will be understood that each block of the flowchart, and combinations of blocks in the flowchart, may be implemented by various means, such as hardware, firmware, processor, circuitry and/or other device associated with execution of software including one or more computer program instructions. For example, one or more of the procedures described above may be embodied by computer program instructions. In this regard, the computer program instructions which embody the procedures described above may be stored by a memory device of a user terminal and executed by a processor in the user terminal. As will be appreciated, any such computer program instructions may be loaded onto a computer or other programmable apparatus (e.g., hardware) to produce a machine, such that the instructions which execute on the computer or other programmable apparatus create means for implementing the functions specified in the flowchart block(s). These computer program instructions may also be stored in a computer-readable memory that may direct a computer or other programmable apparatus to function in a particular manner, such that the instructions stored in the computer-readable memory produce an article of manufacture which implements the functions specified in the flowchart block(s). The computer program instructions may also be loaded onto a computer or other programmable apparatus to cause a series of operations to be performed on the computer or other programmable apparatus to produce a computer-implemented process such that the instructions which execute on the computer or other programmable apparatus implement the functions specified in the flowchart block(s).

Accordingly, blocks of the flowchart support combinations of means for performing the specified functions and combinations of operations for performing the specified functions. It will also be understood that one or more blocks of the flowchart, and combinations of blocks in the flowchart, can be implemented by special purpose hardware-based computer systems which perform the specified functions, or combinations of special purpose hardware and computer instructions.

In this regard, a method according to one embodiment of the invention (as discussed above in reference to FIG. 11) may include receiving first sensor data from a first sensor including a first electrode and a second electrode at operation 1100, where the first sensor is disposed proximate to a chest of a wearer. The method may further include receiving second sensor data from a second sensor including a third electrode and a fourth electrode at operation 1110, where the second sensor is disposed proximate a distal end of a limb of the wearer. The method may further include synchronizing the first sensor data and the second sensor data to extract signal data from the first and second sensor data at operation 1120, performing feature extraction on the first and second sensor data to estimate the biological signal based on comparing results of the feature extraction to a model at operation 1120, and storing a continuous record of the biological signal at operation 1140.

A corresponding system for monitoring a biological signal in a wearable context may also be provided. The system may include a first sensor including a first electrode and a second electrode disposed proximate to a chest of a wearer to obtain first sensor data, a second sensor including a third electrode and a fourth electrode disposed proximate a distal end of a limb of the wearer to obtain second sensor data, and a monitoring device wirelessly operably coupled to the first sensor and the second sensor to receive the first sensor data and the second sensor data. The monitoring device may be time-synchronized with the first and second sensors and include processing circuitry configured to extract signal data from the first and second sensor data. The monitoring device performs feature extraction on the first and second sensor data to estimate the biological signal based on comparing results of the feature extraction to a model. A continuous record of the biological signal is stored by the monitoring device.

In some embodiments, the method, system or an apparatus configured for use with either may include features or operations described above that may be further augmented or modified, or additional features or operations may be added. These augmentations, modifications and additions may be optional and may be provided in any combination. Thus, although some example modifications, augmentations and additions are listed below, it should be appreciated that any of the modifications, augmentations and additions could be implemented individually or in combination with one or more, or even all of the other modifications, augmentations and additions that are listed. As such, for example, the signal data extracted by the monitoring device may include electrocardiogram (ECG) data extracted based on the first sensor data and photoplethysmography (PPG) data extracted based on the second sensor data. In an example embodiment, the signal data extracted by the monitoring device may further include accelerometry, electrodermal activity and electromyography. In some cases, the results of the feature extraction include data corresponding to pulse transit time (PTT), pulse arrival time (PAT) and pre-ejection period (PEP). In an example embodiment, the biological signal includes blood pressure. In some cases, the monitoring device may be operably coupled to the first and second sensors via a low energy data transmission modality (e.g., BLUETOOTH® Low Energy (BLE)). In an example embodiment, the model may include a rational quadratic Gaussian Process Regression (GPR), a quadratic Support Vector Machine (SVM), a fine tree or a linear SVM. In some cases, the model may be updated responsive to accumulation of the continuous record from the wearer and a plurality of continuous records associated with other wearers. In an example embodiment, the model may be normalized across different physical activities based on a comparison of signal data to ground truth measurements made during the different physical activities while building the model. In some cases, the system may further include at least a third sensor, and the monitoring device may be configured to selectively enable and disable different combinations of the first sensor, the second sensor, and the third sensor for estimating the biological signal. In an example embodiment, the monitoring device may be configured to selectively enable and disable different combinations of the first, second, third and fourth electrodes to facilitate measurements between selected combinations of the first, second, third and fourth electrodes to estimate the biological signal. In some cases, the first and second sensors may each be disposed in corresponding sensor holders of a garment, and a battery powering both the first and second sensor includes portions at distributed locations of the garment. In some cases, the monitoring device may also receive additional sensor or healthcare related information from a medical professional or the wearer, and determines a healthcare related risk rating based on the first and second sensor data, the estimated biological signal and the additional sensor or healthcare related information.

In an example embodiment, an apparatus for performing the method described above may include a processor (e.g., the processor 120) or processing circuitry configured to perform some or each of the operations (1100-1140) described above. The processor may, for example, be configured to perform the operations (1100-1140) by performing hardware implemented logical functions, executing stored instructions, or executing algorithms for performing each of the operations. In some embodiments, the processor or processing circuitry may be further configured for the additional operations or optional modifications to operations 1100 to 1140 that are discussed above.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe exemplary embodiments in the context of certain exemplary combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. In cases where advantages, benefits or solutions to problems are described herein, it should be appreciated that such advantages, benefits and/or solutions may be applicable to some example embodiments, but not necessarily all example embodiments. Thus, any advantages, benefits or solutions described herein should not be thought of as being critical, required or essential to all embodiments or to that which is claimed herein. Although specific terms are employed herein, they are used in a generic and descriptive sense only and not for purposes of limitation.

What is claimed is:

1. A system for monitoring a biological signal in a wearable context, the system comprising:
a first sensor comprising a first electrode and a second electrode, the first sensor being disposed proximate to a chest of a wearer to obtain first sensor data;
a second sensor comprising a third electrode and a fourth electrode, the second sensor being disposed proximate a distal end of a limb of the wearer to obtain second sensor data; and
a monitoring device wirelessly operably coupled to the first sensor and the second sensor to receive the first sensor data and the second sensor data,
wherein the monitoring device is time-synchronized with the first and second sensors and comprises processing circuitry configured to extract signal data from the first and second sensor data,
wherein the monitoring device performs feature extraction on the first and second sensor data to estimate the biological signal and generate an estimated biological signal based on comparing results of the feature extraction to a model, and
wherein a continuous record of the biological signal is stored by the monitoring device.

2. The system of claim 1, wherein the signal data extracted by the monitoring device includes electrocardiogram (ECG) data extracted based on the first sensor data and photoplethysmography (PPG) data extracted based on the second sensor data.

3. The system of claim 2, wherein the signal data extracted by the monitoring device further includes accelerometry, electrodermal activity and electromyography.

4. The system of claim 2, wherein the results of the feature extraction include data corresponding to pulse transit time (PTT), pulse arrival time (PAT) and pre-ejection period (PEP).

5. The system of claim 1, wherein the biological signal comprises blood pressure.

6. The system of claim 1, wherein the continuous record is stored long term for analysis to determine and predict health-related aspects of the wearer.

7. The system of claim 6, further comprising an artificial intelligence (AI) module configured to analyze the continuous record to determine stroke risk based on continuous blood pressure estimates.

8. The system of claim 1, wherein the model is updated responsive to accumulation of the continuous record from the wearer and a plurality of continuous records associated with other wearers.

9. The system of claim 1, wherein the model is normalized across different physical activities based on a comparison of signal data to ground truth measurements made during the different physical activities while building the model.

10. The system of claim 1, further comprising at least a third sensor, wherein the monitoring device is configured to selectively enable and disable different combinations of the first sensor, the second sensor, and the at least the third sensor for estimating the biological signal.

11. The system of claim 1, wherein the monitoring device is configured to selectively enable and disable different combinations of the first, second, third and fourth electrodes to facilitate measurements between selected combinations of the first, second, third and fourth electrodes to estimate the biological signal.

12. The system of claim 1, wherein the first and second sensors are each disposed in corresponding sensor holders of a garment, and
wherein a battery powering both the first and second sensor includes portions at distributed locations of the garment.

13. The system of claim 1, wherein the monitoring device receives additional sensor or healthcare related information from a medical professional or the wearer, and determines a healthcare related risk rating based on the first and second sensor data, the estimated biological signal and the additional sensor or healthcare related information.

14. An apparatus for monitoring a biological signal in a wearable context, the apparatus comprising processing circuitry configured to execute instructions that, when executed, cause the apparatus to:
receive first sensor data from a first sensor comprising a first electrode and a second electrode, the first sensor being disposed proximate to a chest of a wearer;
receive second sensor data from a second sensor comprising a third electrode and a fourth electrode, the second sensor being disposed proximate a distal end of a limb of the wearer;
synchronize the first sensor data and the second sensor data to extract signal data from the first and second sensor data;
perform feature extraction on the first and second sensor data to estimate a biological signal based on comparing results of the feature extraction to a model; and
store a continuous record of the biological signal.

15. The apparatus of claim 14, wherein the signal data extracted by the apparatus includes electrocardiogram (ECG) data extracted based on the first sensor data and photoplethysmography (PPG) data extracted based on the second sensor data.

16. The apparatus of claim 15, wherein the signal data extracted by the apparatus further includes accelerometry, electrodermal activity and electromyography.

17. The apparatus of claim 14, wherein the apparatus is operably coupled to the first and second sensors via a low energy data transmission modality.

18. The apparatus of claim 14, wherein the model is updated responsive to accumulation of the continuous record from the wearer and a plurality of continuous records associated with other wearers, and
wherein the model is normalized across different physical activities based on a comparison of signal data to ground truth measurements made during the different physical activities while building the model.

19. The apparatus of claim 14, wherein the processing circuitry is configured to selectively enable and disable different combinations of the first, second, third and fourth electrodes to facilitate measurements between selected combinations of the first, second, third and fourth electrodes to estimate the biological signal.

20. A method for monitoring a biological signal in a wearable context comprising:
receiving first sensor data from a first sensor comprising a first electrode and a second electrode, the first sensor being disposed proximate to a chest of a wearer;
receiving second sensor data from a second sensor comprising a third electrode and a fourth electrode, the second sensor being disposed proximate a distal end of a limb of the wearer;
synchronizing the first sensor data and the second sensor data to extract signal data from the first and second sensor data;
performing feature extraction on the first and second sensor data to estimate the biological signal based on comparing results of the feature extraction to a model; and
storing a continuous record of the biological signal.
